(19) United States
(12) Patent Application Publication
(10) Pub. No.: US 2023/0289496 A1
(43) Pub. Date: Sep. 14, 2023

(54) METHOD OF PREDICTING LOW-CYCLE FATIGUE CRACK INITIATION AND PROPAGATION BEHAVIORS UNDER MULTI-SCALE FRAMEWORK

(71) Applicant: Tianjin University, Tianjin (CN)

(72) Inventors: Lei ZHAO, Tianjin (CN); Lianyong XU, Tianjin (CN); Kai SONG, Tianjin (CN); Yongdian HAN, Tianjin (CN)

(21) Appl. No.: 18/097,912

(22) Filed: Jan. 17, 2023

(30) Foreign Application Priority Data
Mar. 14, 2022 (CN) 202210247708.1

(51) Int. Cl. G06F 30/23

(52) U.S. Cl. CPC G06F 30/23; G06F 2119/02

(57) ABSTRACT

A method of predicting low-cycle fatigue crack initiation and propagation behaviors under a multi-scale framework includes the following steps: S1, providing a calculation method for low-cycle fatigue crack initiation and propagation damages under a multi-scale framework; S2, determining a slip system where a maximum damage is located and an accumulated damage of all slip systems by calculation using the calculation method in S1; S3, a crack initiating and propagating in a direction towards the slip system where the maximum damage is located when the accumulated damage reaches a critical value; and S4, conducting calculation repeatedly until a predicted crack length reaches a fracture length of a low-cycle fatigue specimen under test conditions.

### TECHNICAL FIELD

The present invention relates to the technical field of reliability evaluation, and more specifically, relates to a method of predicting low-cycle fatigue crack initiation and propagation behaviors under a multi-scale framework.

### BACKGROUND ART

As a research on reliability of structural parts deepens, a low-cycle fatigue failure behavior of a material under a cyclic load is an important problem that researchers have to face. Crack initiation and crack propagation are two vital components in the research on low-cycle fatigue failure behaviors. Among them, a crack initiation life accounts for about 40% to 60% of the low-cycle fatigue life of a specimen, and a propagation rate of a crack will fluctuate obviously when the crack changes from short to long. These important phenomena cannot be directly observed in traditional fatigue tests, especially in high-temperature low-cycle fatigue tests. However, in-situ low-cycle fatigue tests have some problems, such as high cost and small specimen dimensions; and there are some limitations when they are related to the crack propagation behaviors of large dimension specimens. Therefore, how to accurately predict the low-cycle fatigue crack initiation and propagation behaviors is of great significance for revealing a fracture mechanism of low-cycle fatigue damages.

A morphology of a low-cycle fatigue fracture presents a typical transgranular fracture characteristic, and a crack having an irregular morphology propagates in a rather random direction. At present, a method of predicting a crack propagation behavior under a low-cycle fatigue condition is mainly based on the extended finite element technique, and it is considered that the direction of crack propagation is consistent with that of a maximum stress/strain. However, this prediction method usually needing to preset a crack in a model cannot predict a crack initiation behavior. Moreover, the assumption that the direction of crack propagation is along the direction of the maximum stress/strain ignores the influence of a microstructure on low-cycle fatigue crack initiation and propagation behaviors. Therefore, developing a method of predicting low-cycle fatigue crack initiation and propagation behaviors under a multi-scale framework by coupling the influence of a micro-slip band on crack initiation and propagation behaviors in a fatigue process to a prediction model is of great significance for revealing the micro-fracture mechanism of low-cycle fatigue.

### SUMMARY

An object of the present invention is to provide a method of predicting low-cycle fatigue crack initiation and propagation behaviors under a multi-scale framework according to the demand of low-cycle fatigue fracture mechanism prediction and development. The method can effectively resolve the limitation that existing prediction methods cannot take into consideration that crack initiation and propagation behaviors are unrelated to a microstructure, and provides a new method for evaluating a fracture mechanism of important parts under a low-cycle fatigue condition.

To attain the above object, the present invention uses a technical solution described hereafter.

A method of predicting low-cycle fatigue crack initiation and propagation behaviors under a multi-scale framework comprises the following steps:
S1, providing a calculation method for low-cycle fatigue crack initiation and propagation damages under a multi-scale framework:

$${\dot{d}}_{initial} = {\sum }_{m} \frac{2 {G ( {γ}_{n , m , e} )}^{2}}{π ( 1 - v ) \cdot {d}^{3} \cdot {w}_{m , critical}} ;$$

$${\dot{d}}_{growth} = {\sum }_{m} ξ {λ}_{d} \frac{\int {τ}_{n , m , e} d {γ}_{n , m , e}}{{w}_{m , critical}} ;$$

wherein, \dot{d}ᵢₙᵢₜᵢₐₗ is a damage rate of low-cycle fatigue crack initiation, \dot{d}_{growth} is a damage rate of low-cycle fatigue crack propagation, m is a number of slip systems, G is a shear modulus, v is the Poisson's ratio, d is an average grain diameter, γ_{n,m,e} is an effective shear strain on slip system m, e is an effective shear stress on slip system m, w_{m,critical} is a fracture energy corresponding to each slip system, ξ is a material parameter, and λ_{d} is a mean free path of dislocations;
S2, determining a slip system where a maximum damage is located and an accumulated damage of all slip systems by calculation using the calculation method in S1;
S3, a crack initiating and propagating in a direction towards the slip system where the maximum damage is located when the accumulated damage reaches a critical value; and
S4, conducting calculation repeatedly until a predicted crack length reaches a fracture length of a low-cycle fatigue specimen under test conditions.

Optionally, the method further comprises: building a low-cycle fatigue finite element model considering a microstructure; and

calculating the effective shear stress and the effective shear strain on different slip systems in each grain by using an orientation of each grain and a macroscopic mechanical response of a material.

Optionally, formulas for calculating the effective shear stress and the effective shear strain are described hereafter:

τ_{n,m,e}=(nₘ)^{T}σₚ(nₘ);

γ_{n,m,e}=(nₘ)^{T}εₚ(nₘ));

wherein nₘ is a normal vector of the slip system m, γ_{n,m} is a shear strain on slip system m, εₚ is a macroscopic plastic strain of the material, and σₚ is a macroscopic plastic stress of the material.

Optionally, the method further comprises: building a fracture energy calculation model under a molecular dynamics system, wherein an XZ plane is defined as a slip plane, a Y direction is defined as a slip direction, and a tensile load at a constant rate is applied in the Y direction.

Optionally, a method of calculating fracture energy of the slip system is as follows:

w_{m,critical}=∫ₛ_{₁}^{s}^{₂}τ_{n,m}ds_{n,m};

wherein s₁ is a displacement corresponding to a peak stress, s₂ is a corresponding displacement when a fracture occurs, s_{n,m} is a tensile displacement on slip system m, and τ_{n,m} is a tensile stress on slip system m.

It can be known from the above technical solution that, compared with the prior art, the present invention provides the calculation method for the low-cycle fatigue crack initiation and propagation damages under the multi-scale framework, which has the following beneficial effects:
1. The present invention is the method of predicting the low-cycle fatigue crack initiation and propagation behaviors under the multi-scale framework, which resolves the limitation of traditional low-cycle fatigue crack prediction, and offers a new thought for revealing the low-cycle fatigue fracture mechanism of the material.
2. The influence of the microstructure (the grain size and the orientation) on fatigue crack initiation and propagation is considered in the present invention.
3. The role of the fracture mechanism of a slip zone in fatigue crack initiation and propagation is considered in the present invention.
4. The crack initiation damage and the crack propagation damage in a low-cycle fatigue process are simultaneously calculated in the present invention.
5. The method of the present invention is proved to have a good effect in predicting a low-cycle fatigue crack.

### BRIEF DESCRIPTION OF THE DRAWINGS

The drawings required for use in the description of the embodiments or the prior art will be briefly described hereinafter in order to more clearly explain the embodiments of the present invention or the technical solution in the prior art. It will be apparent that the drawings described herein are only embodiments of the present invention, and other drawings may be obtained from the drawings provided without any creative effort by those of ordinary skill in the art.

FIG. 1 is a schematic diagram of a research route for a method of predicting low-cycle fatigue crack initiation and propagation behaviors under a multi-scale framework in the present invention.

FIG. 2 is a fracture energy calculation model under a molecular dynamics system and a tensile stress-displacement relation diagram.

FIG. 3 is a schematic diagram of a low-cycle fatigue finite element model considering a microstructure.

FIG. 4 is a schematic diagram of a crack propagation direction criterion in the present invention.

FIG. 5 is a comparison diagram between predicted results of low-cycle fatigue crack length and morphology proposed by the present invention and test results.

FIG. 6 is a comparison diagram between predicted results of a low-cycle fatigue crack fracture slip system family proposed by the present invention and test results.

### DETAILED DESCRIPTION

The technical solutions in the embodiments of the present invention will be clearly and completely described hereafter in conjunction with the accompanying drawings in the embodiments of the present invention, and it will be apparent that the described embodiments are only a part of the embodiments of the present invention rather than all embodiments. Based on the embodiments in the present invention, all other embodiments obtained by those of ordinary skill in the art without any creative effort are within the scope of protection of the present invention.

Referring to FIG. 1, the present invention provides a method of predicting low-cycle fatigue crack initiation and propagation behaviors under a multi-scale framework, comprising: a calculation method for low-cycle fatigue crack initiation and propagation damages under a multi-scale framework, a fracture energy calculation method under a molecular dynamics system, a low-cycle fatigue stress-strain calculation method considering a microstructure, and a crack renewal method based on a low-cycle fatigue condition under the multi-scale framework. The present invention will make further explanation by a low-cycle fatigue test and finite element simulation, wherein G115 martensitic heat-resistant steel is used as a test material, the test temperature is 650° C., the low-cycle fatigue test adopts strain loading, and a loading waveform is triangular.

In step (1), the calculation method for the low-cycle fatigue crack initiation and propagation damages under the multi-scale framework is provided.

$${\dot{d}}_{initial} = {\sum }_{m} \frac{2 {G ( {γ}_{n , m , e} )}^{2}}{π ( 1 - v ) \cdot {d}^{3} \cdot {w}_{m , critical}}$$

$${\dot{d}}_{growth} = {\sum }_{m} ξ {λ}_{d} \frac{\int {τ}_{n , m , e} d {γ}_{n , m , e}}{{w}_{m , critical}}$$

wherein, \dot{d}^{initial} is a damage rate of low-cycle fatigue crack initiation, \dot{d}_{growth} is a damage rate of low-cycle fatigue crack propagation, m is a number of slip systems, G is a shear modulus, v is a Poisson's ratio, d is an average grain diameter, γ_{n,m,e} is an effective shear strain on slip system m, τ_{n,m,e} is an effective shear stress on slip system m, w_{m,critical} is a fracture energy corresponding to each slip system, ξ is a material parameter, and λ_{d} is a mean free path of dislocations. Firstly, crack initiation damages begin to accumulate under a fatigue load. When a value of an accumulated low-cycle fatigue crack initiation damage is equal to 1, a crack initiates. After that, a low-cycle fatigue crack propagation damage starts to be calculated. When the value of an accumulated low-cycle fatigue crack propagation damage is equal to 1, the crack propagates.

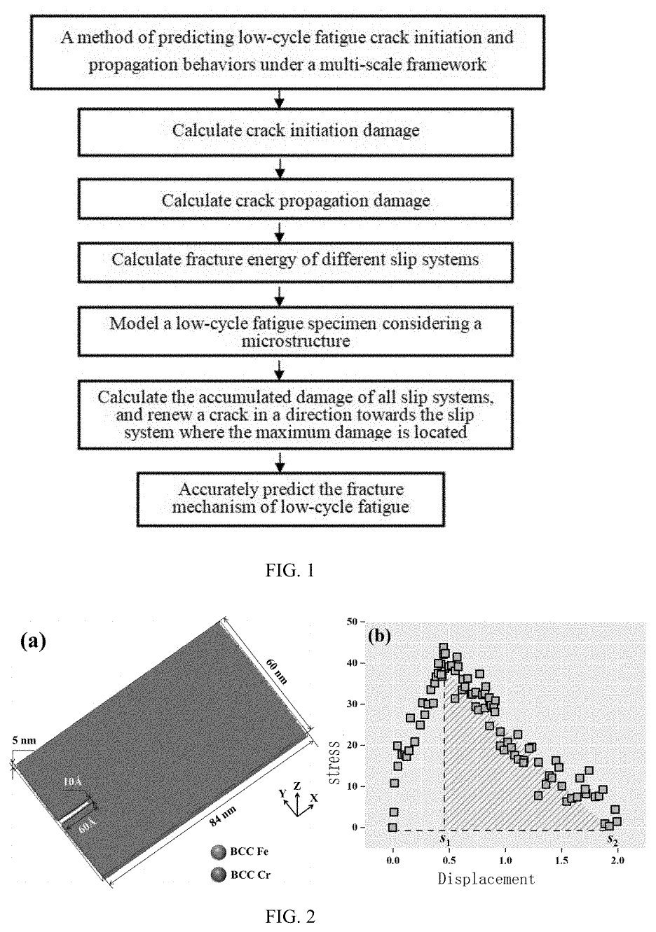

In step (2), a fracture energy calculation model under a molecular dynamics system is built, wherein an XZ plane is defined as a slip plane, a Y direction is defined as a slip direction, and a tensile load at a constant rate is applied in the Y direction, as is illustrated in FIG. 2(a). It should be ensured in the fracture energy calculation model that the lengths in X and Y directions are greater than 20 nₘ, and the length in a Z direction is greater than 4 times of a lattice constant. Calculation methods for fracture energies of different slip systems are put forward according to the law of a stress-displacement curve, as is illustrated in FIG. 2(b).

w_{m,critical}=∫ₛ_{₁}^{s}^{₂}τ_{n,m}ds_{n,m}

wherein s₁ is a displacement corresponding to a peak stress, s₂ is a corresponding displacement when fracture occurs, s_{n,m} is a tensile displacement on slip system m, and τ_{n,m} is a tensile stress on slip system m.

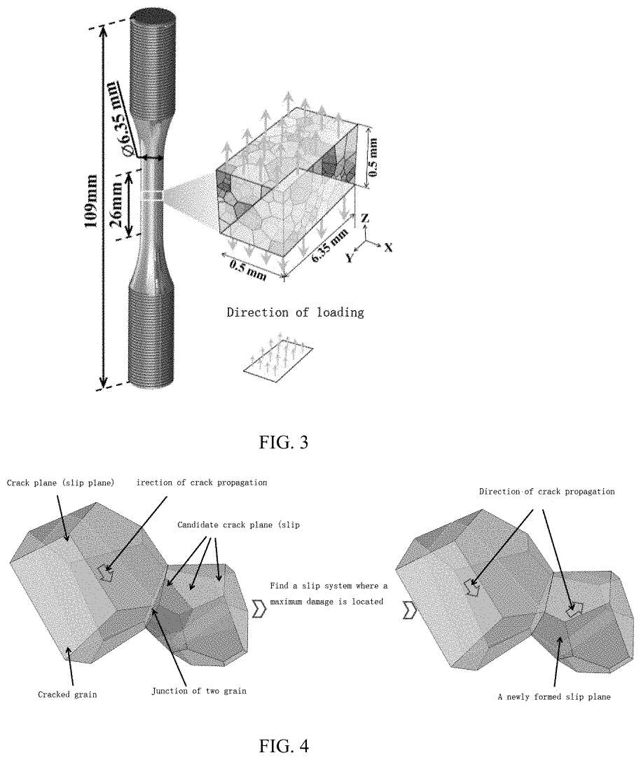

In step (3), a low-cycle fatigue finite element model considering a microstructure is built, as is illustrated in FIG. 3. The model has a length consistent with a diameter of a specimen, and a loading direction thereof is consistent with that in the test. The effective shear stress and effective shear strain on different slip systems in each grain are calculated by using an orientation of each grain and a macroscopic mechanical response of a material.

τ_{n,m,e}=(nₘ)^{T}σₚ(nₘ) γ_{n,m,e}=(nₘ)^{T}εₚ(nₘ)

wherein, nₘ is a normal vector of slip system m, γ_{n,m} is a shear strain on slip system m, εₚ is a macroscopic plastic strain of the material, and σₚ is a macroscopic plastic stress of the material. Effective shear stress/strain is a projection of a plastic stage in macroscopic stress/strain on slip system m.

In step (4), an accumulated damage of grains under a cyclic load is calculated using the formulas in steps (1) to (3). When a value of the accumulated crack initiation/propagation damage reaches 1, the slip system where a maximum damage is located is determined by using the formula in step (1). A crack usually breaks along the slip system under the low-cycle fatigue condition. Therefore, a predicted crack is renewed along the slip system where the maximum damage is located. FIG. 4 is a schematic diagram of a crack propagation criterion in the method.

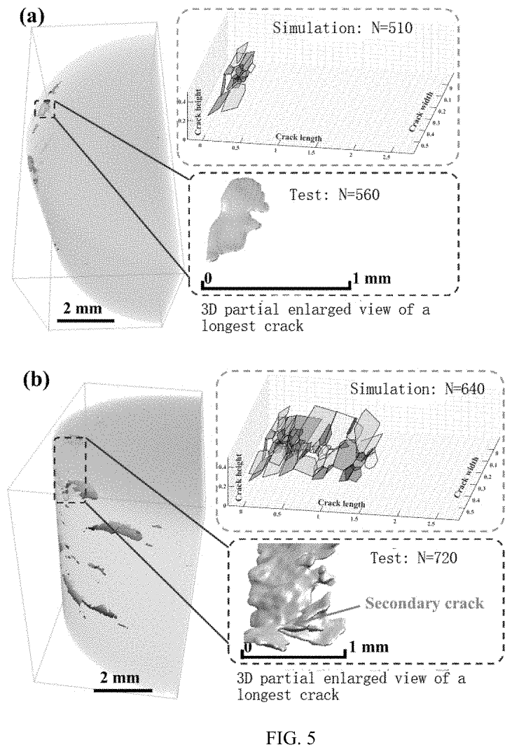

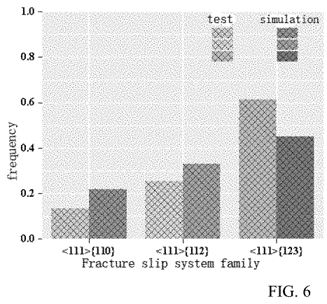

In step (5), steps (1) to (4) are executed repeatedly until a predicted crack length reaches a fracture length of a low-cycle fatigue specimen under test conditions. It is found by observing the fracture of G115 steel in a low-cycle fatigue test at 650° C. that the test stops when the crack propagates to 3 mm. In order to verify the effect of the method of predicting the low-cycle fatigue crack initiation and propagation behaviors based on the multi-scale framework provided by the present invention, low-cycle fatigue crack prediction results, obtained by the method, of the G115 steel at 650° C. are compared with test results, as is illustrated in FIG. 5 and FIG. 6. The prediction results and the test results show high accuracy in characterizing the crack length and a proportion of a fracture slip system family. Therefore, the method of predicting the low-cycle fatigue crack initiation and propagation behaviors under the multi-scale framework provided by the present invention can well predict a low-cycle fatigue fracture mechanism.

The embodiments in the description are described in a progressive manner and each embodiment focuses on differences from the other ones. Identical and similar parts of the embodiments can be referred to by one another. Apparatuses disclosed in the embodiments are described relatively simple since they correspond to the methods disclosed in the embodiments, and the relevant parts can refer to the description of the methods.

The above description of the disclosed embodiments enables those skilled in the art to practice or use the present invention. Various modifications to these embodiments will be apparent to those skilled in the art and the general principles defined herein may be implemented in other embodiments without departing from the spirit or scope of the present invention. Therefore, the present invention will not be limited to these embodiments shown herein but shall accord with the widest scope consistent with the principles and novel features disclosed herein.

1. A method of predicting low-cycle fatigue crack initiation and propagation behaviors under a multi-scale framework, characterized by comprising the following steps:
S1, providing a calculation method for low-cycle fatigue crack initiation and propagation damages under a multi-scale framework:
$${\dot{d}}_{initial} = {\sum }_{m} \frac{2 {G ( {γ}_{n , m , e} )}^{2}}{π ( 1 - v ) \cdot {d}^{3} \cdot {w}_{m , critical}} ;$$
$${\dot{d}}_{growt ▯} = {\sum }_{m} {ξλ}_{d} \frac{\int {τ}_{n , m , e} d {γ}_{n , m , e}}{{w}_{m , critical}} ;$$
wherein \dot{d}ᵢₙᵢₜᵢₐₗ is a damage rate of low-cycle fatigue crack initiation, \dot{d}_{growth} is a damage rate of low-cycle fatigue crack propagation, m is a number of slip systems, G is a shear modulus, v is a Poisson's ratio, d is an average grain diameter, γ_{n,m,e} is an effective shear strain on the slip systems m, τ_{n,m,e} is an effective shear stress on the slip systems m, w_{m,critical} is a fracture energy corresponding to each slip system, ξ is a material parameter, and λ_{d} is a mean free path of dislocations;
S2, determining a slip system where a maximum damage is located and an accumulated damage of all the slip systems m by calculation using the calculation method in S1;
S3, a crack initiating and propagating in a direction towards the slip system where the maximum damage is located when the accumulated damage reaches a critical value; and
S4, conducting calculation repeatedly until a predicted crack length reaches a fracture length of a low-cycle fatigue specimen under test conditions.

2. The method of predicting the low-cycle fatigue crack initiation and propagation behaviors under the multi-scale framework according to claim 1, further comprising: building a low-cycle fatigue finite element model considering a microstructure; and calculating the effective shear stress and effective shear strain on different slip systems of the slip systems m in each grain by using an orientation of each grain and a macroscopic mechanical response of a material.

3. The method of predicting the low-cycle fatigue crack initiation and propagation behaviors under the multi-scale framework according to claim 1, wherein formulas for calculating the effective shear stress and effective shear strain are described below:
τ_{n,m,e}=(nₘ)^{T}σₚ(nₘ),
γ_{n,m,e}=(nₘ)^{T}εₚ(nₘ);
wherein nₘ is a normal vector of the slip systems m, γ_{n,m} is a shear strain on the slip systems m, εₚ is a macroscopic plastic strain of a material, and σₚ is a macroscopic plastic stress of the material.

4. The method of predicting the low-cycle fatigue crack initiation and propagation behaviors under the multi-scale framework according to claim 1, further comprising: building a fracture energy calculation model under a molecular dynamics system, wherein an XZ plane is defined as a slip plane, a Y direction is defined as a slip direction, and a tensile load at a constant rate is applied in the Y direction.

5. The method of predicting the low-cycle fatigue crack initiation and propagation behaviors under the multi-scale framework according to claim 1, wherein a method of calculating the fracture energy of the slip system is as follows:
w_{m,critical}=∫ₛ_{₁}^{s}^{₂}τ_{n,m}ds_{n,m};
wherein s₁ is a displacement corresponding to a peak stress; s₂ is a corresponding displacement when a fracture occurs; s_{n,m} is a tensile displacement on the slip systems m; and τ_{n,m} is a tensile stress on the slip systems m.

6. The method of predicting the low-cycle fatigue crack initiation and propagation behaviors under the multi-scale framework according to claim 1, wherein the low-cycle fatigue specimen is martensitic heat-resistant steel, and the test conditions comprise: adopting strain loading with a loading waveform being triangular at a target test temperature.

7. The method of predicting the low-cycle fatigue crack initiation and propagation behaviors under the multi-scale framework according to claim 1, further comprising:
evaluating reliability of the low-cycle fatigue specimen based on a predicted result obtained by the S4.
